(19) United States
(12) Patent Application Publication
(10) Pub. No.: US 2010/0010611 A1
(43) Pub. Date: Jan. 14, 2010

(54) STENT BASED METHOD AND APPARATUS FOR DIRECTING EXTERNAL BEAM RADIATION THERAPY

(75) Inventor: Frank Johnson, St. Louis, MO (US)

Correspondence Address: HUSCH BLACKWELL SANDERS LLP, 190 Carondelet Plaza, Suite 600, ST. LOUIS, MO, 63105, US

(73) Assignee: SAINT LOUIS UNIVERSITY, St. Louis, MO (US)

(21) Appl. No.: 12/170,070

(22) Filed: Jul. 9, 2008

(51) Int. Cl. A61F 2/84; A61F 2/82; A61N 5/00; A61B 6/08

(52) U.S. Cl. 623/1.11; 623/1.34; 623/23.7; 378/205; 600/433

(57) ABSTRACT

A stent based apparatus and method for directing external beam radiation comprising a series of devices and a system of implanting these devices within a patient to direct therapeutic radiation given to the patient. Each of the devices can deliver a tiny gold fiducial (or fiducial constructed of another material capable of acting as a target to direct external beam therapeutic radiation) to a target area in the vicinity of a cancer or other lesion that is considered an appropriate target for external beam radiation therapy, using, for example, the beam radiation system referred to as the Cyberknife radiation delivery system. The current system and devices envision placement of gold fiducials through hollow organs in the body (for example, the gastrointestinal tract, the ureter, blood vessels, bile duct, or the cerebro spinal fluid space). This implantation method can utilize conventional endoscopy or angiography.

### BACKGROUND OF INVENTION

1. Field of Invention

This invention relates generally to beam radiation therapy and, more particularly, to targets for directing beam radiation therapy.

2. Background Art

There are various devices that can be utilized to assist in directing a radiation beam to a point of incident or target area in the vicinity of a cancer or other lesion during therapeutic radiation treatment given to a patient having such cancer or lesion. If a cancer or lesion is considered an appropriate target for external beam radiation therapy, often a device referred to as a fiducial is implanted in or near the target area of the patient. The system providing the radiation beam can have a tracking system that is able to locate the fiducial, define a coordinate for the location of the fiducial, and redirect or pan the radiation beam to focus on the coordinate defined by the location of the fiducial. The fiducials can typically be constructed of gold or other material appropriate for the tracking system. In some instances, placement of gold fiducials can be done easily using simple implant techniques such as placement during surgical exploration, implantation using a large-bore needle guided by computer assisted tomography scan (such as for example CT), and other percutaneous approaches.

As the concept of implanted devices for external beam targeting has evolved, numerous permutations on the fiducial marker concept have arisen. The most frequently utilized devices in most series have been gold seeds, visualizing them using a variety of imaging methodologies. Typically, the seeds are cylindrical, allowing ease of insertion via a large-bore needle with guidance by an imaging system like ultrasound. They generally have surface features that prevent seed migration after insertion. Varying sizes of seed marker, including custom lengths, may be ordered. Carbon fiducials hold the promise of visibility on CT and producing less artifact as compared with gold.

The markers are implantable devices designed to act as reliable surrogates for imaging anatomic structures of interest. Fiducial marker techniques were originally developed in the pre-conformal radiotherapy era for positional verification of tissues that were not easily visualized using portal X-ray film imaging for patient alignment. Soft tissue structures that were relatively mobile could be more readily seen when radio-opaque seeds or wires were implanted in or near organs of interest.

The present invention relates to systems and methods for implanting devices. Radiation therapy is used to treat localized cancers and other lesions or conditions. There are various examples of radiation therapy treatments including conventional external beam radiation therapy, as well as three-dimensional conformal external beam radiation, intensity modulated radiation therapy (IMRT), a “gamma knife” that employs highly focused gamma ray radiation obtained by crossing or collimating several radiation beams, stereotactic radiosurgery and brachytherapy. The success of the radiation treatment can directly depend on the total dose of radiation delivered to the target region. However, the amount of radiation effectively delivered to the target region (as well as the amount delivered to adjacent healthy tissue) can vary from a desired or planned amount. This is particularly problematic when radiation therapy is used on deep tumors, or when the therapy is delivered to tumors located close to healthy radiation sensitive regions or organs. Typically, the radiation therapy is directed not only to the known tumor location, but also to healthy tissue adjacent to the tumor based on a predetermined treatment margin. The more imprecise the radiation delivery is thought to be, the larger the planned treatment margin that may be used.

Radiation can obviously be detrimental to healthy tissue. Therefore, a therapy goal is to use smaller treatment margins while delivering radiation doses in desired amounts and to the desired target with precision. However, delivering external beam radiation doses in the desired dose amount to the actual tumor site can be complicated as the tumor may shift over time, either during or between radiation sessions. That is, in certain locations in the body, such as in the prostate, movement of target tissue may occur during radiation treatment, primarily attributable to the patient's breathing or filling and/or emptying of the bladder. Thus, dynamic changes in the position of the tumor during active radiation delivery can increase the potential of collateral damage to healthy or non-targeted tissue.

Using positional markers to determine spatial positioning information has been part of the solution for targets located deep within a patient's body. Such localization may be used to direct or guide the radiation therapies. Such a system defines a target isocenter and the sensors in the external array and compares the location of the target isocenter with the machine isocenter before and during radiation therapy.

Existing methods and systems for implanting fiducials have various short-comings. The implantation methods of many are difficult to perform and often fiducials move once inserted. The present invention will address these as well as other problems with existing technology.

### BRIEF SUMMARY OF INVENTION

The invention is a stent-based method and apparatus for directing external beam radiation. It comprises a series of devices and a system of implanting these devices to direct therapeutic radiation given to a patient. Each of the devices described can deliver a gold fiducial (or fiducial constructed of another compatible material) to a target area in the vicinity of a cancer or other lesion that is considered an appropriate target for external beam radiation therapy using, for example, the beam radiation system referred to as the Cyberknife radiation delivery system. The current system and devices envision placement of gold fiducials through hollow organs in the body (for example, the gastrointestinal tract, the ureter, blood vessels, bile duct, or the cerebro spinal fluid space). This implantation method could utilize conventional endoscopy or angiography devices.

The devices can carry the gold fiducial and fix it in place as a permanent implant. The technique of the present invention can be useful for directing the application of other forms of electromagnetic or other radiation such as highly focused microwave beam, heavy particle radiation, and so forth. There is a widely recognized need for, and it would be highly advantageous to have, a system and devices for directing the placement of fiducials within a patient's body.

Using naturally occurring spaces and channels, endoscopes, and therapeutic tools introduced through endoscopes, are ubiquitous in modern medical practice. Similarly, vascular access to even very small vessels can now be done routinely using modern interventional radiological methods. Thus far, introduction of fiducials through these approaches has not been feasible. The present inventions would successfully address the shortcomings of presently available fiducial placement techniques by providing a system and devices which enable the clinician to place fiducials in pre-selected spots using natural channels and existing placement devices and techniques.

The first category of devices of the present invention can comprise modifications of existing stents (vascular, intestinal, biliary). These stents were originally designed to maintain narrowed areas of the above named structures in an open position. Often this narrowing is due to a tumor or other disease process that might be amenable to external beam electromagnetic radiation. Placement of fiducials by means of these stents would be a convenient way to maintain them within the target. It is anticipated that the composition of the stent would minimize other radio opaque material in order to avoid problems with detection of the gold fiducial created by a metallic stent wall. The stents could be covered or uncovered, depending on the situation and therapeutic considerations.

A secondary category of devices of the present invention can be intended to fill a small vessel, anchoring the fiducial with spring-loaded prongs. This would be introduced through a vascular catheter and thus can be similar to a number of products now used to occlude or stunt a hollow vessel. The devices considered in this application would be anchored in place within the vessel lumen.

A third category of devices of the present invention can be modifications of pre-existing enteric stents. They can be introduced by an endoscope. These could be covered or uncovered with fabric and would contain the usual amount of metallic material (if stenting were required) or a minimal amount of metallic material (if therapeutic stenting were not a treatment goal). More prongs than usual could be used to fix these stents in place.

A fourth category of devices of the present invention can be introduced through a neurosurgical endoscope or placed at the time of surgery on the central nervous system. These would attach gold fiducials to a clip, applied by a specially created application device to a denticulate ligament or other suitable structure.

One embodiment of implanting a fiducial for directing external beam radiation can comprise the steps of providing an endoscope having an elongated tube portion with a leading insertion end and a tail end, including a lengthwise hollowed channel. An inner tube can be telescopically extended through the lengthwise hollowed channel. A fiducial deployment mechanism similar to those known in the art can be attached proximate the insertion end of the inner tube. A fiducial having at least a portion of its structure constructed of a radio opaque material can be provided for the deployment mechanism. The elongated tube portion can be extended through a natural body channel of a patient. The fiducial can be implanted with the fiducial deployment mechanism at a target location within the natural body channel. Tracking and identifying a coordinate of the radio opaque material of the fiducial can be performed with an external beam radiation system operable for directing the external beam to focus on the volume defined by the coordinates. The fiducial can be a stent that can be expanded in the natural body channel.

For example, a patient with a tumor situated in the presacral area near the iliac arteries or veins could be localized for external beam radiation using stents in the iliac artery or vein, possibly in combination with other fiducials introduced through other techniques. Another example is a biliary tumor in which external beam radiation therapy is contemplated, whereby the present method could be implemented having fiducial implants using a conventional biliary stent modified to incorporate the tiny gold marker. Yet another example is a lesion in the spinal canal whereby the present method could be utilized such that the lesion could be marked using ventriculoscope through a conventional surgical incision and anchored in placed using a clip on a neurologically insignificant structure.

These and other advantageous features of the present invention will be in part apparent and in part pointed out herein below.

### BRIEF DESCRIPTION OF THE DRAWINGS

For a better understanding of the present invention, reference may be made to the accompanying drawings in which:

FIG. 1 is an illustration of a typical endoscope;

FIG. 2 is an illustration of an endoscope inserted through the esophagus into and through the stomach into the duodenum of a patient to a cancer site;

FIG. 3 is an illustration of an expanded stent deployed;

FIGS. 4a and 4b are an illustration of a stent fiducial;

FIG. 5 is an illustration of a pronged fiducial; and

FIG. 6 is an illustration of an alternative embodiment pronged fiducial.

While the invention is susceptible to various modifications and alternative forms, specific embodiments thereof are shown by way of example in the drawings and will herein be described in detail. It should be understood, however, that the drawings and detailed description presented herein are not intended to limit the invention to the particular embodiment disclosed, but on the contrary, the intention is to cover all modifications, equivalents, and alternatives falling within the spirit and scope of the present invention as defined by the appended claims.

### DETAILED DESCRIPTION OF INVENTION

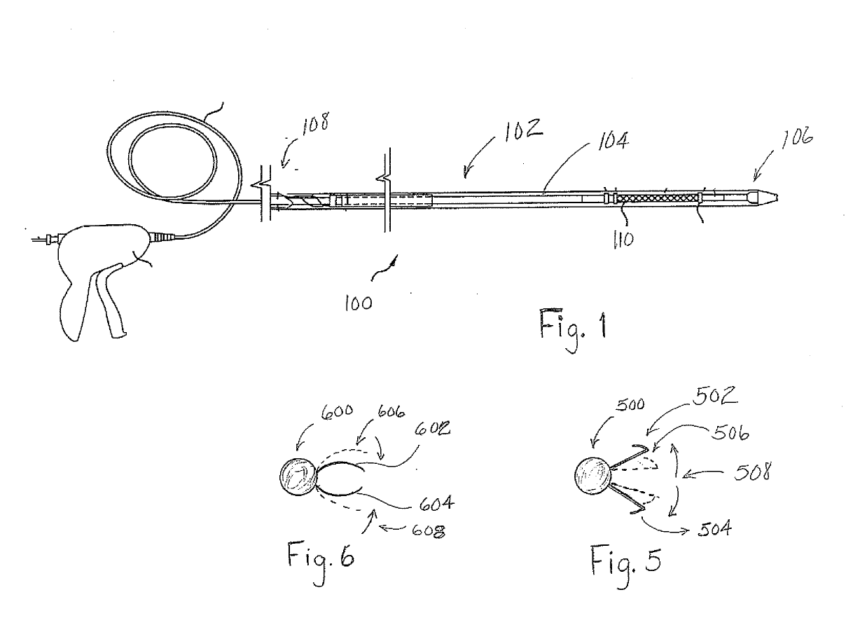

According to the embodiment(s) of the present invention, various views are illustrated in FIGS. 1-6 and like reference numerals are being used consistently throughout to refer to like and corresponding parts of the invention for all of the various views and figures of the drawing. Also, please note that the first digit(s) of the reference number for a given item or part of the invention should correspond to the Fig. number in which the item or part is first identified.

The present invention relates to the fields of endoscopy and angiography and utilizes similar tools as these fields. Angiography involves a physical intervention in an artery. A common example is a narrowed coronary artery which may benefit from treatment to reduce the risk of a heart attack. One option to reduce this risk is a coronary intervention, such as balloon angioplasty or a stent. Interventional cardiologists perform angioplasty, which opens narrowed arteries. One method is to use a long, thin tube called a catheter that has a small balloon on its tip. The balloon can be inflated at the blockage site in the artery to flatten or compress the plaque against the artery wall. Angioplasty is also called percutaneous transluminal coronary angioplasty (PTCA).

A stent is a small, mesh-like device made of metal. When a stent is placed inside of a coronary artery, it acts as a support, keeping the vessel open. By keeping the vessel open, the stent helps to improve blood flow to the heart muscle and reduce the pain of angina. Stent procedures can be used along with balloon angioplasty. Physicians can insert and thread the catheter through a peripheral artery into a patient's coronary artery. A video monitor (like a TV screen) can be used to see the process. Once the catheter reaches the narrowed or blocked artery, a harmless dye is injected, and the physician will take a picture of the coronary arteries (called a coronary angiogram). The angiogram helps the physician see the size and location of the blockage.

The stent can be put at the tip of the catheter, over the balloon or other device operable to release or expand the stent. When the catheter is positioned at the blockage, the balloon is inflated or some other expansion means is utilized, expanding the stent. The catheter and guide wire are then removed, leaving the stent behind to hold the artery open. One embodiment of the present invention modifies the stent and insertion technique to effectively direct a radiation beam to the desired target area during external beam radiation therapy.

In a similar manner, endoscopic procedures are performed by inserting and threading an endoscope through a body channel, such as, for example, the colon. For example, the physician performing the procedure can insert and thread a tube called an endoscope into a patient's mouth and passing it down the patient's esophagus to the stomach. An endoscope can be described as a flexible telescope. The endoscope allows the physician to see inside the patient's body using the eye piece at the top of the tube or by watching a screen. The physician can pass the endoscope tube all the way down through the stomach to the duodenum until it reaches the opening of the bile duct. Once it is in place, the physician can inject a dye into the bile duct and can use an X-ray to watch the flow of the dye and see where the duct is blocked. Once the doctor has found the blockage, he or she can dilate the duct and put the stent in place. One embodiment of the present invention can also utilize an endoscope inserted through a body channel.

One embodiment of the present invention comprising a stent-based device for directing external beam radiation involves a novel apparatus and method for providing a target to direct a radiation beam to a specific location during radiation therapy. For this embodiment the stent can be modified with a special radio-opaque material in a small area of the stent or the entire stent can be constructed of the radio-opaque material if possible. Another embodiment of the invention can utilize other fiducial designs, such as for example, fiducials having prongs for attaching to tissue to hold the fiducial at a target location.

The details of the invention and various embodiments can be better understood by referring to the figures of the drawing. Referring to FIG. 1, an illustration of a typical endoscope is shown. A typical endoscope 100 can have an elongated tube portion 102 that can be flexible. The elongated tube portion can be designed for insertion into a natural body canal such as, for example, a bronchus or esophagus. The flexible tube portion 102 of the endoscope can have an interior hollowed channel 104, which extends from the leading end 106 to the trailing end 108. There can be one or more inner tubes telescopically extending through the channel 104 for performing various functions. Various cables can also extend through the tubing in order to perform various functions or to mechanically manipulate and guide the leading end 106. When the leading end 106 is appropriately positioned within a natural body channel or cavity, the inner tubes can be telescopically extended beyond the leading end 106 for deployment of various devices, such as a stent as illustrated by item number 110.

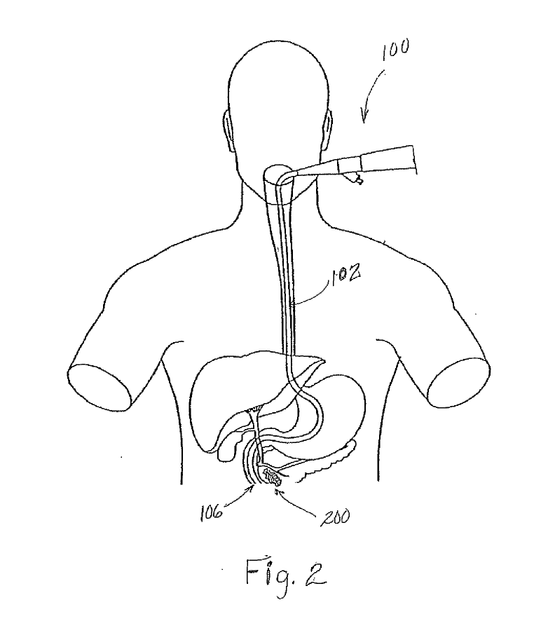

Referring to FIG. 2, an illustration of an endoscope 100 inserted through the esophagus into and through the stomach into the duodenum of a patient to a cancer site is shown. The tube portion 102 can be threaded through the natural body channels and cavities until the insertion end of the tube is located at a desired site. FIG. 2 illustrates the insertion end 106 extended to a location adjacent a cancer 200. The inner tubes of the endoscope can be extended to deploy a fiducial that can later be utilized for directing a radiation beam utilized during external beam radiation therapy. The fiducial can be constructed of gold or some other radiopaque material that is capable of being utilized to direct external beam radiation. The inner tube can be designed to carry the fiducial and fix it in place as a permanent implant to be later utilized for directing the beam radiation. Once the implant is in place, it can be utilized for directing not only beam radiation, but other forms of electromagnetic beams such as for example, highly focused microwave beams or heavy particle radiation. Using naturally occurring spaces and channels, endoscopes and angioscopes utilized in combination with other therapeutic tools introduced through the endoscope or angioscope can be accomplished utilizing modern medical practices. Similarly, vascular access can be accomplished utilizing modern intervential radiological methods. The present invention successfully addresses the shortcomings of presently available placement techniques for fiducials by providing a system and device which places fiducials in pre-selected spots utilizing natural body channels and existing placement devices and techniques.

Referring to FIG. 3, an illustration of an expanded stent is shown. Stents of this type can be utilized in the vascular channel such as arterial channels. Larger stents can be utilized in other body channels such as, for example, in the intestinal tract or in the urethra in the area of the prostate. Stents of this type can be utilized to reopen a natural body channel to its normal diameter. Stents of this type can be deployed by way of an endoscope device.

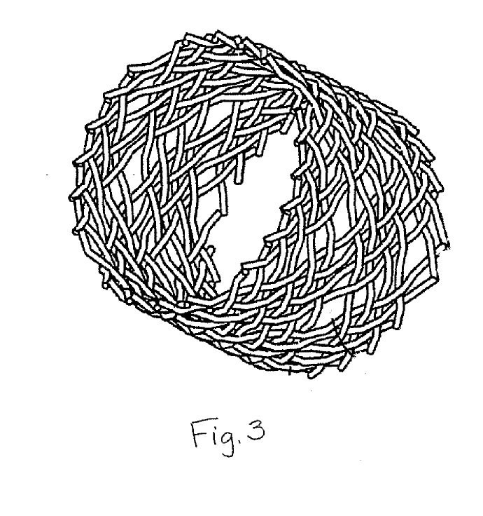

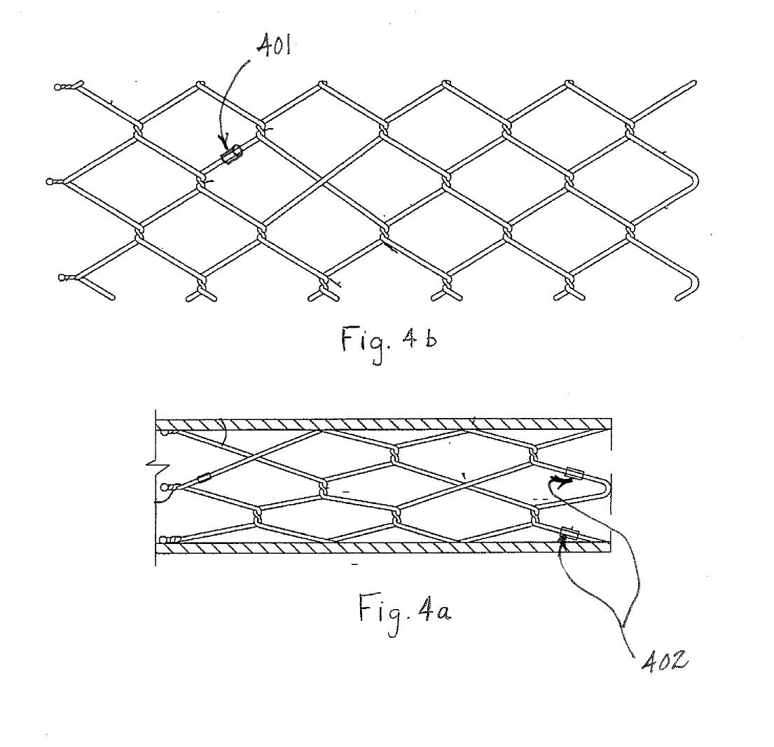

Referring to FIGS. 4a and 4b, an illustration of a stent fiducial is shown. FIG. 4a illustrates a side section of a stent installed in a vascular channel such as an artery. This side section is also illustrated in FIG. 4b. This section of the stent can be constructed radiopaque of a radiopaque material such as, for example, gold, such that the stent can be utilized for directing a radiation beam utilized doing external beam radiation therapy. The embodiment shown in FIGS. 4A and 4B show fiducials 401 and 402 attached to the stent. Once deployed, the stent fiducial is a permanent implant that can be utilized repetitively during external radiation therapy.

Referring to FIG. 5, an illustration of a pronged fiducial is shown. This pronged fiducial 500 is an alternative embodiment of a fiducial implant. The fiducial 500 can have two prongs 502 and 504, each having hook members for attaching to internal body tissue in order to hold the fiducial in place. Prior to deployment, the prongs can be compressed to a position illustrated by broken lines 506 and when deployed, the prongs can expand outward as indicated by directional arrows 508. The fiducial 500 can be contained within a tube prior deployment. The tube can have a diameter substantially equivalent to the diameter of the diameter of the fiducial such that the prongs 502 and 504 are compressed inward to a position indicated by broken lines 506. The tube containing the fiducial can have a mechanism for extending beyond the insertion end of the endoscope for deployment of the fiducial. When the prongs extend outward, they can attach and mount to the surrounding tissue utilizing the hooks of the prongs.

Referring to FIG. 6, an illustration of an alternative embodiment pronged fiducial is shown. The fiducial 600 is yet another embodiment of a fiducial that can be utilized in the present invention. Again, the fiducial can have two prongs 602 and 604 that are utilized to attach to tissue in the target area. Prior deployment, the prongs of the fiducial can be expanded outward as indicated by broken lines 606. When deployed, the prongs of the fiducial can be released and retract toward each other as indicated by a directional arrow 608. Again, the fiducial 600 can be extended beyond the insertion end of the endoscope and deployed in the tissue surrounding the target area. The prongs 604 and 606 can act as a clip device that attaches to a ligament or other suitable tissue structure.

The various stent-based target examples shown above illustrate a novel method and apparatus for providing a target to direct a radiation beam to a specific location when being emitted during radiation therapy. A user of the present invention may choose any of the above stent-based target embodiments, or an equivalent thereof, depending upon the desired application. In this regard, it is recognized that various forms of the subject stent-based target invention could be utilized without departing from the spirit and scope of the present invention.

As is evident from the foregoing description, certain aspects of the present invention are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the sprit and scope of the present invention.

Other aspects, objects and advantages of the present invention can be obtained from a study of the drawings, the disclosure and the appended claims.

What is claimed is:

1. A method of implanting a fiducial for directing external beam radiation comprising the steps of:
providing an endoscope having an elongated tube portion with a leading insertion end and a tail end and including a lengthwise hollow channel;
extending an inner tube telescopically through the lengthwise hollow channel;
providing a fiducial deployment mechanism operably attached proximate to the insertion end of the inner tube;
providing a fiducial having at least a portion of its structure constructed of a radiopaque material;
extending the elongated tube portion through a natural body channel of a patient;
implanting the fiducial with the fiducial deployment mechanism at a target location within the natural body channel; and
tracking and identifying a coordinate of the radiopaque material of the fiducial with an external radiation beam tracking system and directing the external radiation beam focused on the coordinate.

2. The method for implanting a fiducial as recited in claim 1, where the step of providing a fiducial is providing a stent.

3. The method for implanting a fiducial as recited in claim 2, further comprising the step of expanding the stent in the natural body channel.

4. The method for implanting a fiducial as recited in claim 3, where the step of extending is extending into arterial channel.

5. The method for implanting a fiducial as recited in claim 1, where the step of providing a fiducial is providing a fiducial with a main body constructed of a radiopaque material and having prongs extending away from the main body beyond an outermost diameter of the main body, and further comprising the steps of:
compressing the prongs inward within the outermost diameter prior implanting and releasing the prongs outward extending beyond the outermost diameter for anchoring the fiducial in surrounding tissue.

6. The method for implanting a fiducial as recited in claim 1, where the step of providing a fiducial is providing a fiducial with a main body constructed of a radiopaque material and having prongs extending away from the main body and within an outermost diameter of the main body, and further comprising the steps of:
expanding the prongs outward beyond the outermost diameter prior implanting and releasing the prongs to retract inward one with respect to the other extending within the outermost diameter for anchoring the fiducial in surrounding tissue.

7. An apparatus for implanting a fiducial for directing external beam radiation comprising the steps of:
an endoscope having an elongated tube portion with a leading insertion end and a tail end and having a sufficiently small diameter for insertion in a natural body channel and including a lengthwise hollow channel;
an inner elongated member extending telescopically through the lengthwise hollow channel;
a fiducial deployment mechanism operably attached proximate the insertion end of the inner elongated member having deployably mounted there to a fiducial having at least a portion of its structure constructed of a radiopaque material; and
an external radiation beam tracking system having a radiopaque material tracking system operable for tracking and identifying a coordinate of the radiopaque material of the fiducial when implanted in a patient and further operable to direct the external beam to focus on the coordinate.

8. The apparatus for implanting a fiducial as recited in claim 7, where the fiducial is a stent.

9. The apparatus for implanting a fiducial as recited in claim 8, where the stent is cylindrical and operable to expand radially outward from its cylindrical axis within the natural body channel.

10. The apparatus for implanting a fiducial as recited in claim 9, where the endoscope is diametrically sized for extending into an arterial channel.

11. The apparatus for implanting a fiducial as recited in claim 7, where the fiducial has a main body constructed of a radiopaque material and having prongs extending away from the main body beyond an outermost diameter of the main body, and designed to be compressed inward within the outermost diameter prior implanting and to be expanded outward extending beyond the outermost diameter when released for anchoring the fiducial in surrounding tissue.

12. The apparatus for implanting a fiducial as recited in claim 7, where the fiducial is a fiducial with a main body constructed of a radiopaque material and having prongs extending away from the main body and within an outermost diameter of the main body, and the prongs operable to be expanded outward beyond the outermost diameter prior implanting prior implanting and to retract inward one with respect to the other extending within the outermost diameter when released for anchoring the fiducial in surrounding tissue.
